(19) United States
(12) Patent Application Publication
(10) Pub. No.: US 2016/0374850 A1
(43) Pub. Date: Dec. 29, 2016

(54) Genital Shield for Reducing the Transmission of Sexually Transmitted Diseases

(71) Applicant: Arif H. Agha, Oakbrook, IL (US)

(72) Inventor: Arif H. Agha, Oakbrook, IL (US)

(21) Appl. No.: 15/261,889

(22) Filed: Sep. 10, 2016

Related U.S. Application Data
(63) Continuation-in-part of application No. 14/100,318, filed on Dec. 9, 2013.

(51) Int. Cl. A61F 6/06

(52) U.S. Cl. CPC A61F 6/06

(57) ABSTRACT

This invention relates to a genital shield capable of being coupled with a condom for use in the perineal or labial area of a female. This shield comprises an adhesive dressing for application on a user's genital region that is bio-occlusive, thin, flexible, unobtrusive to the senses, and aesthetically pleasing to a user.

### CROSS-REFERENCE TO RELATED APPLICATIONS

This application is a continuation in part of U.S. application Ser. No. 14/100,318 filed on Dec. 9, 2013 entitled “Genital Shield for Reducing the Transmission of Sexually Transmitted Diseases”.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LIST COMPACT DISC APPENDIX

Not applicable.

### BACKGROUND OF THE INVENTION

Well before current modern times, people have endeavored to prevent pregnancy and disease with various forms of barrier type devices that shielded one's genitalia from contact with a partner's. Today, common, effective barrier devices include such devices as the male or female condoms and dental dams. Male condoms, in particular, are a popular form of using a barrier to prevent the spread of disease and to reduce the rate of an unwanted pregnancy. Users have largely found condoms to be unobtrusive in terms of sensation or aesthetic appeal which adds to their popularity. While barrier devices have been successful in reducing disease and pregnancy, there is still a risk of spreading disease as condoms or other barrier devices do not cover all of a person's possible infected area. This is especially true of diseases such as genital herpes, genital warts (Human Papilloma Virus; “HPV”), syphilis, chanchroid, and other diseases that are spread through skin to skin contact and can be found on the genito-scrotal and suprapubic area of a male and/or the labial and perineal area of a female. As such there have been many attempts to cover this area of the body to help reduce the spread of disease.

One known solution to this problem is seen in U.S. Pat. No. 4,834,114 (Boarman) issued on May 30, 1989 and U.S. Pat. No. 5,113,873 (Boarman) issued on May 19, 1992. This relates to a contraceptive system similar in design to the male condom but including a genital shield member. A releasable fastener mechanism secures the shield to the individual. This invention is for use by females as the condom like portion is inserted into the vagina and the shield is releasably attached to the pubic area of the female. This problem with these known solutions is that the releasable fastener mechanism tends to have poor adherence qualities which results in lowered protection against disease transmission.

Another known solution is seen in U.S. Pat. No. 5,413,117 (Wills) issued on May 9, 1995. This invention relates to a prophylactic device having a shield member that lies over the pubic region. This device is held onto a user's body by adjustable straps. One known problem with this solution is that the adjustable straps of this invention are not viewed positively by users as there is friction and movement with the straps and the straps are not aesthetically pleasing.

Another known solution is seen in U.S. Pat. No. 5,168,881 (Reddy) issued on Dec. 8, 1992. This relates to a prophylactic device which is intended to be worn as a garment and further is able to provide a shield for either a male or female perineum for added protection against disease. This device also utilizes straps for holding the device in place which causes friction and unwelcome movement of the device and the garment can be unappealing to a user.

Another solution is seen in U.S. Pub. No. 20100071702 (Stuhrlingh). This relates to a condom and molded shield portion that rests against a user's pubic and scrotum area. A ring retains the condom portion over the penis. An issue with this type of solution is that a molded portion rarely fits all users as in order to be effective, the mold would have to properly fit against a user's body. Since bodies vary greatly in size, the molding would not fit all body types properly and would need to be custom made. Further, moldings tend to be semi-rigid or hard by nature and this would significantly reduce sensation during intercourse for a user.

All of the above known prior art solutions attempted to solve the issue of creating an effective barrier against disease but none were able to do it in an unobtrusive, aesthetically pleasing manner that appropriately fit a user without unintended movement. The issue with any device providing a barrier in the genital area is that this area of the body has non-conforming anatomy, there are issues of moisture, poor adherence against the skin (sometimes due to hair), loss of coverage due to friction or moisture, and decreased sensation in users. Further, many of these solutions require that the proscribed condom be used with the devices instead of allowing a user preference in condom choice which is known to be an important feature in the popularity of barrier shields. This invention solves all of these problems by utilizing an adhesive film dressing as a shield that is known to adhere well to skin, does not diminish sensation or aesthetic appeal, is flexible enough to adhere to any body type, and allows a user to use his/her preferred condom in conjunction with the shield.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### BRIEF SUMMARY OF THE INVENTION

This invention relates to a genital shield for use in conjunction with a condom to aid in reducing the spread of sexually transmitted disease. More specifically it relates to a shield comprising a bio-occlusive, aesthetically pleasing barrier that does not reduce sensation that is applied to the genital region where a condom does not protect. The barrier comprises a dressing having an adhesive and non-adhesive side wherein the dressing is coupled to a backing sheet on the adhesive side and a border on the non-adhesive side.

In its preferred embodiment, the dressing comprises a thin, flexible, bio-occlusive material. Since this dressing will be used by persons engaging in sexual intercourse, a preferred dressing must also be aesthetically pleasing to all of a user's senses. The dressing must be thin enough so as to not interfere with sensations on the skin. A preferred material will also be transparent; however one skilled in the art will recognize that translucent, semi-transparent, or solid materials may be anticipated. In this type of use, the more transparent a dressing is the more aesthetically pleasing it may be considered.

Such preferred dressing material may include material such as transparent film dressings commonly used in wound care or to dress surgical sites. There are a wide variety of dressing materials marketed under a variety of names such as Tegaderm® (3M), Bioclusive® (Johnson & Johnson Company), Hydrofilm® (Paul Hartmann Aktiengesellschaft), Opsite® (T. J. Smith & Nephew), Suresite® (Medline Industries), or Mepore® (Molnlycke Health Care AB Corporation). This list is not intended to be inclusive of the types of surgical wound dressings available and only serves to list examples of possible known dressings that hold characteristics that would be desirable to be used in the dressings of this invention. These film dressings are known to be highly effective in preventing wound or surgical infection in patients while giving patients a thin, flexible, unobtrusive dressing. Patients are able to wear these dressings for an extended period of time without the dressing separating from the skin and allowing for a user to continue normal activities without significant restriction. The dressings does not allow bacteria or viruses to be introduced to the skin area it is covered when used properly. Such material would translate well into the material for this dressing as the material provides a fluid-proof bacterial or viral barrier against the skin. The thin, flexible, bio-occlusive nature of this material makes it a preferred choice of material for the dressing of this invention.

The dressing of this invention is contoured to fit the female anatomy, namely the labial and perineal regions. One skilled in the art will recognize that the dressing may be in a variety of sizes to meet the needs of a population with a variety of body sizes.

The dressing further comprises an adhesive side coupled to a backing sheet. The adhesive side of the dressing is to be applied to the body after it is peeled away from the backing sheet. The backing sheet is preferred to be a stiff sheet of paper, plastic, or other suitable material. The dressing is peeled away from this backing sheet in a manner similar to a sticker being removed from its backing sheet. A user then applies the dressing to the external skin of the labial and perineal regions with the adhesive side down in the appropriate area. One skilled in the art will recognize that the preferred manner of adhering the dressing to the skin will be achieved when it is applied to dry skin free of hair. While it is possible to adhere the dressing to wet skin or to areas covered with hair, this situation may interfere with the effectiveness of the barrier as its effectiveness relies on securely adhering to one's skin.

The dressing further comprises a non-adhesive side coupled to a border sheet having tabs. This border sheet allows the dressing to maintain its shape and overall integrity after it is removed from the stiff backing sheet. Without the border sheet, the dressing would be extremely difficult to place appropriately on a body due to the thin, flexible nature of the dressing. The border sheet is anticipated to be a thin border around the non-adhesive side to the dressing comprising tabs to allow for easy removal of the border sheet. It is anticipated to be just wide enough to give the dressing shape and integrity during application but thin enough that the dressing can still be seen. It is important that a user be able to see the dressing during application as any error in placing the dressing may cause the dressing to not lay flat and adhere properly against the skin. During application, the user will be able to see how the dressing is adhering to the skin and be able to correct for any misplacement, bubbles or folds in the dressing to ensure the appropriate placement and fit. After the dressing is adhered to the skin, the border sheet is to be removed.

The non-adhesive side of the dressing is preferred to have a smooth texture so as to not be obtrusive or diminish a user's sensations while wearing this dressing.

The dressing contouring to the female anatomy may include labial flaps. These flaps are simply an extension of the dressing and are similarly coupled to a stiff sheet on their adhesive side and a border sheet on the non-adhesive side. As the flaps are removed from the stiff backing sheet, they are then wrapped inwardly around the outer labia. The border sheet is removed after the flaps are appropriately placed. Tabs may be utilized for ease of border sheet removal.

This invention is anticipated to be used in conjunction with an appropriately sized condom. If a female partner is wearing the shield of this invention, then it is anticipated that her male partner will wear a condom. While it is anticipated that only one partner, generally the affected partner, will wear the shield of this invention, one skilled in the art will recognize that both partners can wear the shield of this invention at the same time.

It is anticipated that the nature of this material will allow for extended wear of the shield. A user can apply the shield in advance of a sexual encounter and can continue to wear the shield after the sexual encounter. While it is preferred that the shield be changed in between sexual encounters, it can be worn for multiple encounters between the same partners. If sexual encounters between different partners are anticipated, then the shield must be removed and changed before an encounter with a new partner in order to reduce the risk of disease transmission.

The shield of this invention may be removed from the skin of a user by simply peeling the dressing away from the body. The dressing may then be thrown away.

This shield is anticipated to lay flat while in packaging prior to use.

It is anticipated that this shield will comprise a test patch so that a user can test for sensitivities to this dressing. A user will be able to test in a small area of the body before applying it to a larger area.

This and other embodiments will be more thoroughly realized in the drawings and detailed description below.

### BRIEF DESCRIPTION OF THE SEVERAL VIEWS OF THE DRAWINGS

Possible and preferred features of the present invention will now be described with particular reference to the accompanying drawings. However, it is to be understood that the features illustrated in and described with reference to the drawings are not to be construed as limiting on the broad scope of the invention. In the drawings:

FIG. 1 is an exploded view of the shield contoured to the female anatomy, depicting the dressing, backing sheet, and border sheet, including labial flaps.

FIG. 2 is a perspective view of the shield contoured to the female anatomy without labial flaps.

FIG. 3 is a perspective view of the shield contoured to the female anatomy including the labial flaps.

FIG. 4 is a perspective view of the dressing contoured to the female anatomy including the labial flaps.

FIG. 5 is a perspective view of the dressing as applied to the female anatomy.

FIG. 6 is a cross-sectional view of the dressing as applied to the female anatomy.

FIG. 6A is cross-sectional view of the shield depicting the labial flaps in use.

### DETAILED DESCRIPTION OF THE INVENTION

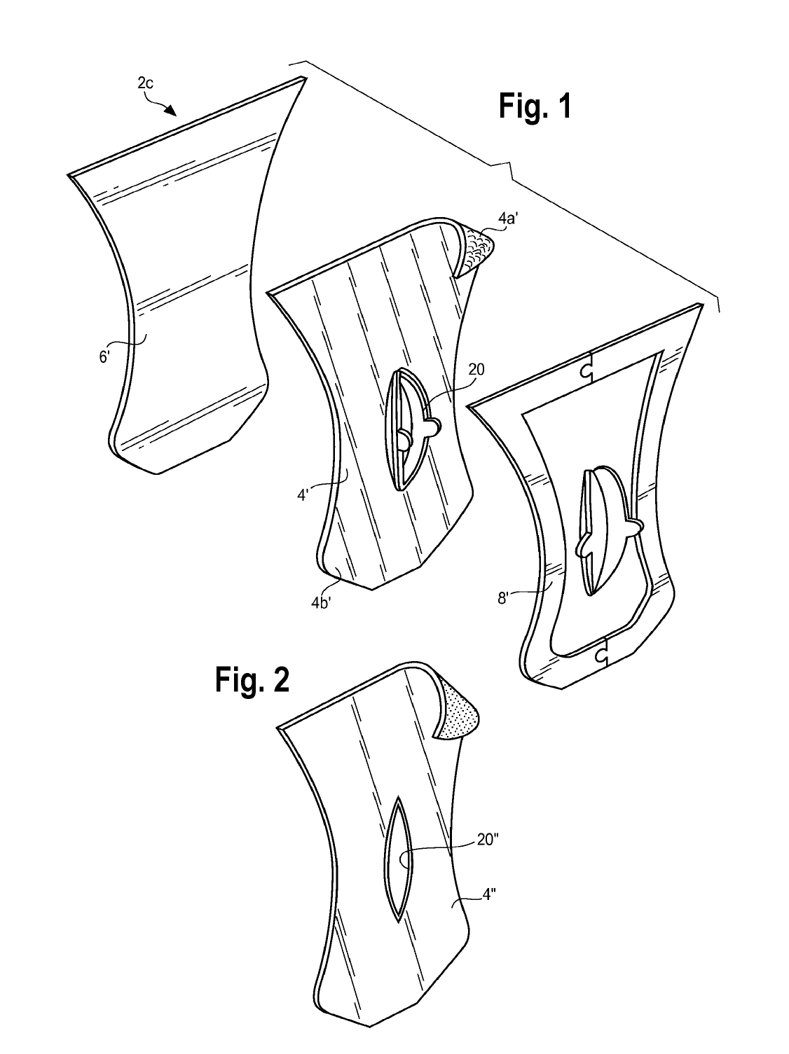

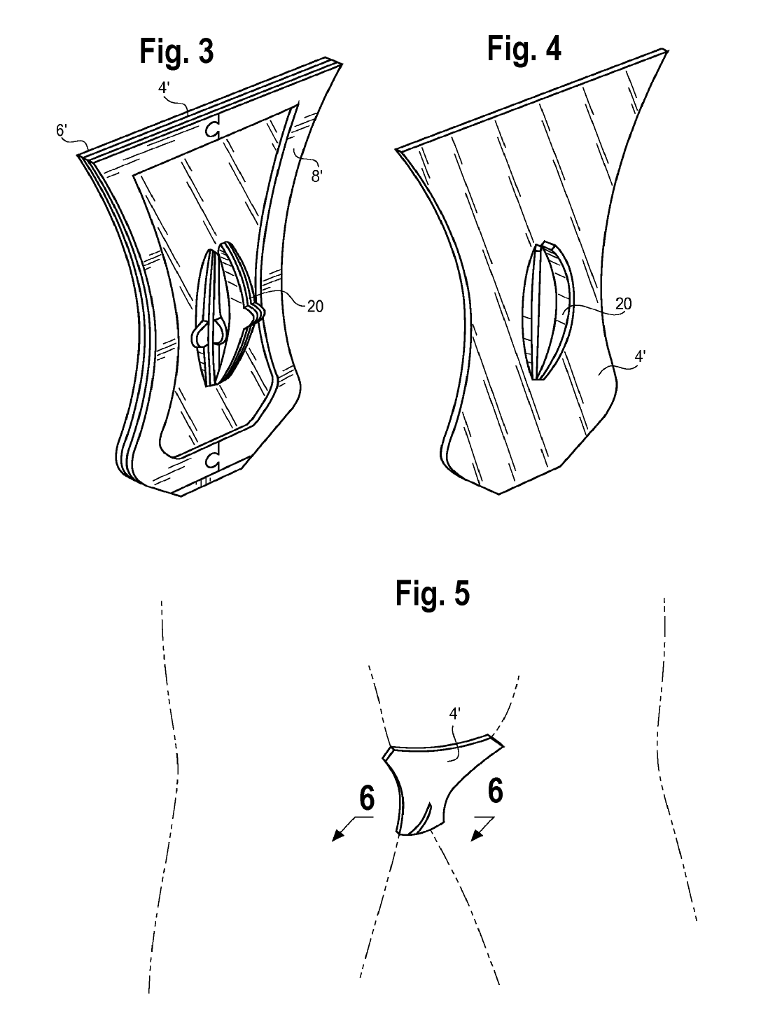

FIGS. 1-6A relate to a barriers 2 contoured to the female anatomy. FIG. 1 depicts an exploded view of the barrier 2, including the backing sheet 6, dressing 4, and border sheet 8 having tabs 9a, 9b. FIG. 1 also depicts the barrier 2 including labial flaps 20. Labial flaps 20 include labial backing sheet 21 and labial border sheet 23 as seen in FIG. 3. However, equally anticipated is the alternate arrangement seen in FIG. 2 without the labial flaps 20. The labial flaps 20 will fold to lay flat while inside of packaging before use.

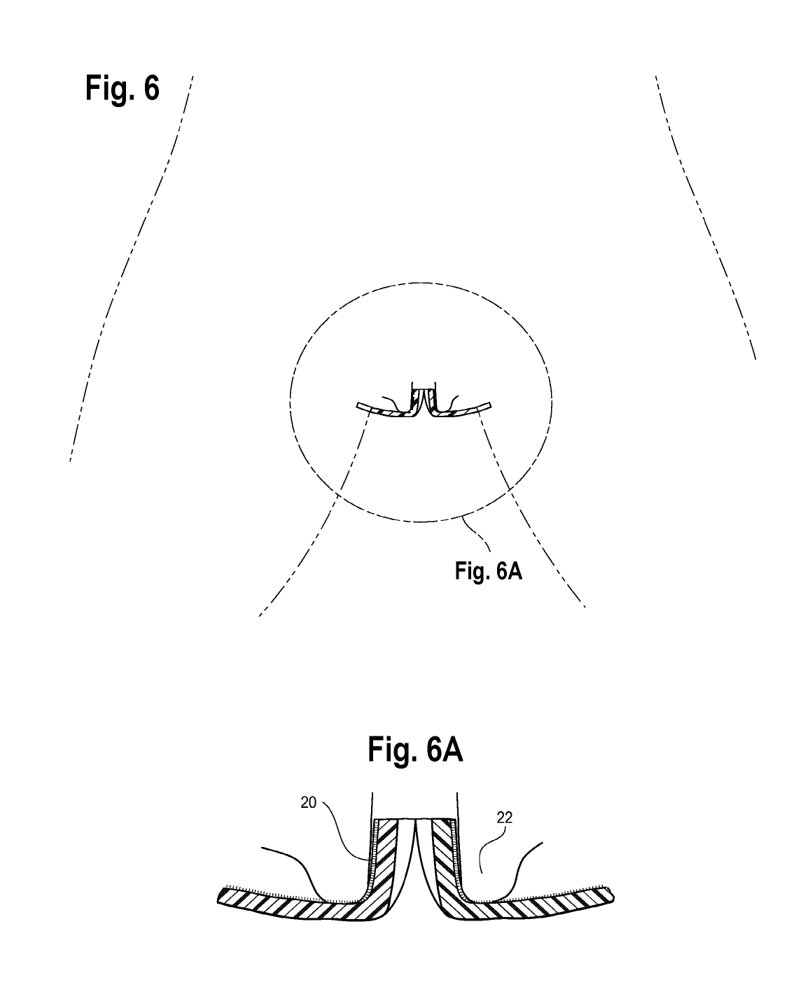

The dressing 4 of the female barrier 2 comprises an adhesive side 4a and a non-adhesive side 4b. The adhesive side 4a of the dressing 4 is joined to the backing sheet 6 and the non-adhesive side 4b is joined to the border sheet 8. To effectively use the barrier 2 contoured to the female anatomy; a user will peel the dressing 4 away from the backing sheet 6 and apply the adhesive side 4a to the user's skin. Once the dressing 4 is applied to the skin over the female labial and/or perineal region, the border sheet 8 can be peeled away exposing the non-adhesive side 4b of the dressing. To assist in peeling away the border sheet 8, tabs 9a, 9b may be utilized. In cases when labial flaps 20 are to be used, the dressing 4 will wrap inwardly around the skin of the labia 22 as seen in FIGS. 6 and 6A. The border sheet 8 will be removed after the labial flaps 20 are adhered appropriately to the labia 22. It is anticipated that for the most protection against disease, that the male partner of the female using this barrier 2 will also use a condom.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

It will be appreciated by those skilled in the art that many modifications and variations may be made to the methods of the invention described herein without departing from the spirit and scope of the invention.

What is claimed is:

1. An external genital shield for application on the female labial and perineal regions for use in conjunction with a male condom to aid in the reduction of the spread of sexually transmitted diseases comprising:
at least one barrier including a dressing; said dressing having an adhesive side and non-adhesive side; said dressing is adapted to conform to female labial and perineal regions;
wherein the dressing is coupled to a backing sheet on the adhesive side and a border sheet with tabs on the non-adhesive side;
wherein the dressing adapted to conform to the external skin of the female labial and perineal regions is adapted to the external skin by removing the dressing from the backing sheet and adhering the dressing to the external skin of the female labial and perineal area; after the dressing is adapted to adhere to the external skin said border sheet is removed.

2. The shield as in claim 1, wherein the dressing adapted to conform to female anatomy further comprises labial flaps.

3. A method for applying an external genital shield to the female labial and perineal regions for use in conjunction with a male condom to aid in the reduction of the spread of sexually transmitted disease comprising the steps of:
applying at least one barrier to the external skin of the labial and perineal regions; said barrier including a body shape conforming dressing wherein said dressing comprises an adhesive side and non-adhesive side; said dressing conforming to the external skin of the labial and perineal regions on a female; and wherein the dressing is coupled to a backing sheet on the adhesive side and a border sheet with tabs on the non-adhesive side;
removing the dressing from the backing sheet and adapting the dressing to adhere to the dressing to the skin of the female labial and perineal regions; and
after adapting the dressing to adhere to the external, skin removing the border sheet by way of the tabs.

4. The shield as in claim 10, wherein the dressing conforming to the female anatomy further comprises labial flaps and further comprises the step of adhering the labial flaps to the labia.
